(19) United States
(12) Patent Application Publication
(10) Pub. No.: US 2023/0167404 A1
(43) Pub. Date: Jun. 1, 2023

(54) Urine-Derived Mesenchymal Stem Cell Mitochondria as Well as Transplantation Method and Use Thereof

(71) Applicant: Peking University People's Hospital, Beijing (CN)

(72) Inventors: Huan Shen, Beijing (CN); Zhixin Jiang, Beijing (CN); Cheng Shi, Beijing (CN); Yanbin Wang, Beijing (CN); Xi Chen, Beijing (CN); Hongjing Han, Beijing (CN); Min Fu, Beijing (CN); Jia Fei, Beijing (CN)

(21) Appl. No.: 17/574,634

(22) Filed: Jan. 13, 2022

(30) Foreign Application Priority Data
Nov. 26, 2021 (CN) 202111418863.7

(51) Int. Cl. C12N 5/075; C12N 5/0775

(52) U.S. Cl. CPC C12N 5/0609; C12N 5/0668

(57) ABSTRACT

A urine-derived mesenchymal stem cell mitochondria as well as a transplantation method and use thereof. The urine-derived mesenchymal stem cell mitochondrion is extracted from urine to be used for improving the quality of oocytes. The transplantation method comprises: jointly injecting sperms and the urine-derived mesenchymal stem cell mitochondria into mature oocytes for blastaea culture during the intracytoplasmic sperm microinjection. The present disclosure has the beneficial effects: during the traditional ICSI in combination with the transplantation of the urine-derived mesenchymal stem cell mitochondria, the fertilization rate of human in-vitro fertilization and the quality of embryos are significantly improved; the urine-derived mesenchymal stem cell mitochondria of the present disclosure can be used for in-vitro fertilization of low-prognosis patients with infertility with a good treatment effect; the problem of an autologous mitochondrion source in the prior art is solved without involving the introduction of a third-party genetic material and ethical issues.

### CROSS REFERENCE TO RELATED APPLICATION

This application claims the benefit of and takes priority from Chinese Patent Application No. 202111418863.7 filed on Nov. 26, 2021, the contents of which are herein incorporated by reference.

### TECHNICAL FIELD

The present disclosure belongs to the technical field of biological medicines, relates to urine-derived mesenchymal stem cell mitochondria as well as a transplantation method and use thereof, and particularly relates to a transplantation method of urine-derived mesenchymal stem cell mitochondria during the intracytoplasmic sperm injection (ICSI) in combination with an autologous non-invasive source.

### BACKGROUND

Under the broad environment where the country's fertility continuously declines and a childbearing age is continuously pushed back, solving the fertility problem of infertile women is a research hotspot in the field of reproduction at present. In vitro fertilization/Intracytoplasmic sperm injection (IVF/ICSI) in an assisted reproductive technology is currently an effective measure to treat infertility, which has a success rate of about 40% internationally, but still has a post-treatment poor pregnancy outcome for the elderly patients or people with poor repeated egg/embryo quality, that is, low-prognosis people. There have been no effective treatment measures for these people so far, and it is needed to develop treatment methods. The current literature evidences prove that the poor quality of oocytes or embryos in this group of people is mainly because of mitochondria. As an important organelle, the mitochondria provide an important energy source for the development of oocytes and embryos. The aging of eggs of elderly patients is often accompanied by decrease in the quantity of mitochondria and reduction in biological function. The abnormality of these mitochondria can lead to insufficient energy supply in the mitotic process of oocytes and increase in aneuploidy rates, and ultimately cause a series of problems such as poor embryo quality and decreased fertility. Therefore, improvement of the prognosis of these people by improving the mitochondria is a clinically feasible treatment manner.

As early as the end of the 20th century, some European countries implemented a method where partial cytoplasmic mitochondria of young people were transplanted into the oocytes of elderly women to treat the infertility of elderly women. This method has achieved remarkable curative effects. By this technology, sperms and about 1%-5% of egg cytoplasm from young donors are jointly injected into recipient oocytes during the ICSI so as to improve the egg quality of patients, especially elderly women. From 1997 to 2001, about 30 children were born as a result of this technology. However, due to the introduction of an allogeneic third-party genetic material, this technology involves ethical and genetic safety issues.

Due to heterogeneity and ethical issues in allogeneic mitochondrion transplantation, an autologous mitochondrion transplantation therapy has recently received widespread attentions. This technology is that autologous cell derived mitochondria are extracted and meanwhile oocytes are injected during the ICSI to improve the infertility problems of their own advance age or poor oocyte quality. Currently, there are few cell sources reported by documents for autologous mitochondrion transplantation, mainly including ovarian stem cells (OSC) and germline granular cells (GC), but these two types of cells are both derived from an ovarian germline, and have the phenomenon of secondary aging accompanied by advanced age. In addition, OSC needs to obtain a large piece of extracted ovarian cortex via surgery, which undoubtedly causes a second injury to patients with a poor ovarian function. Moreover, the existence of OSC in adults has been controversial all the time. Many evidences point out there is no OSC in adults. Therefore, searching the best autologous mitochondrion derived donor sources is a direction that is necessary to deeply study.

Stem cells are similar to those at the egg stage due to their pluripotency and metabolic methods, theoretically, stem cell-derived mitochondria are more suitable for improving the quality of oocytes and embryos, but there are few reports on the basic and clinical research of autologous stem cell mitochondrion transplantation Animal experiments in 2017 found that the transplantation of autologous adipose-derived mesenchymal stem cells (ASC) mitochondria into eggs can significantly improve the developmental potential of old mice eggs. In 2018, Liang Xiaoyan's team from Sun Yat-sen University reported the first case of using autologous bone marrow mesenchymal stem cell (BMSC)-derived mitochondrion transplantation to treat a patient with repeated infertility failures and a successful live birth of a male baby.

Urine-derived mesenchymal stem cells (USC) are adult stem cells with strong proliferation activity and multidirectional differentiation potential, which are non-invasively obtained, isolated and cultured from urine. They have not only biological characteristics of MSC but also the advantages of the mesenchymal stem cell source in the genitourinary system. In addition, USC is isolated from urine, so it has the characteristics of safety and non-invasiveness, unrestricted source, large quantity and simple preparation process and the like, and therefore has become an ideal seed cell in cell replacement therapy and tissue engineering research. A number of studies have applied it to tissue and organ repair and reconstruction and disease model construction. However, there have been no reports on the use of urine-derived mesenchymal stem cells to treat female infertility.

In view of this, this patent is applied.

### SUMMARY

In order to solve the problems existing in the prior art, the present disclosure provides urine-derived mesenchymal stem cell mitochondria as well as a transplantation method and use thereof. Through the transplantation of the urine-derived mesenchymal stem cell mitochondria, the fertilization rate and embryo quality of human in-vitro fertilization have been significantly improved.

The objective of the present disclosure is to provide urine-derived mesenchymal stem cell mitochondria.

Another objective of the present disclosure is to provide a transplantation method of the above urine-derived mesenchymal stem cell mitochondria.

Yet another objective of the present disclosure is to provide use of the above urine-derived mesenchymal stem cell mitochondria.

The technical solution adopted by the present disclosure is as follows:

Provided are urine-derived mesenchymal stem cell mitochondria, which are extracted by the following method:

(1) collecting urine into a vessel, centrifuging, discarding supernatant, adding PBS buffer into the vessel for resuspension, centrifuging again, discarding the supernatant, resuspending cell precipitates using a urine-derived mesenchymal stem cell isolation culture medium, inoculating the resuspended cell precipitates into a gelatin-coated 6-well plate, and putting the 6-well plate into an incubator for primary culture; digesting with pancreatin after cell clones are formed, resuspending using a urine-derived mesenchymal stem cell amplification culture medium after digestion, inoculating into a new 6-well plate, and marking a P1 generation (hereinafter passage and culture are the same as those in this step);

(2) putting the P1 generation obtained in step (1) in the incubator to continue culture, digesting using pancreatin when the P1 generation grows to cover the 85%-95% of area of the 6-well plate, sucking the pancreatin after digestion, resuspending using the urine-derived mesenchymal stem cell amplification culture medium, centrifuging, discarding the supernatant, adding a cell lysis solution, lysing on ice, then adding a mitochondrion extraction solution, and uniformly mixing; and centrifuging, sucking the supernatant, transferring into another vessel, centrifuging again, and discarding the supernatant to obtain precipitates namely mitochondria; and

(3) after step (2), centrifuging again, washing, discarding the supernatant, resuspending the mitochondrion precipitate obtained in step (2) using a mitochondrion preservation solution, and storing at 0-4° C.

Further, in step (1), the centrifuging is performed for 10 min at 120 rpm.

Further, when the cell clones are formed, the culture medium is completely changed once. After the clones are fused into a blockbuster (filled with the entire 100×microscope field), pancreatin is used for digestion, the pancreatin is sucked after digestion, cells are resuspended using the urine-derived mesenchymal stem cell amplification culture medium, inoculated into the new 6-well plate and marked as the P1 generation (subsquent passage and culture are the same as those herein).

More further, in step (1), the time for cell clone formation is 7 days, and the time of fusing the clones into the blockbuster is 14 days.

Further, in step (1), the concentration of gelatin is 0.1%; in step (1) and step (2), the incubators are all incubators with 37° C. and 5% CO₂; the pancreatin is a 0.05% pancreatin aqueous solution; and the digestion time is 1 min.

Further, in step (2), the lysis time is 5 min; the redundant pancreatin is sucked after digestion with pancreatin, 1 mL of urine-derived mesenchymal stem cell amplification culture medium is added into each well for resuspension, the above resuspension is transferred into a centrifuge tube to be centrifuged for 3 min at 1200 rpm, the supernatant is discarded, 500 ul of cell lysis solution is added and lyzed on ice for 5 min, then 1 ml of mitochondrion extraction solution is added and uniformly mixed.

Further, in step (2), 1 ml of mitochondrion extraction solution is added and uniformly mixed; the mixture is centrifuged for 10 min at 800 g, the supernatant is sucked and transferred to another centrifuge tube, and then centrifuged for 10 min at 5000 g, and the supernatant is discarded to obtain precipitates namely mitochondria.

Further, in step (3), the mixture is centrifuged for 10 min at 5000 g and washed, the supernatant is discarded, the mitochondrion precipitate in the centrifuge tube is resuspended using 50 ul of mitochondrion preservation solution, and then the resuspended product is stored at 4° C. or on ice until use.

The transplantation method of the above urine-derived mesenchymal stem cell mitochondria comprises: during the intracytoplasmic sperm microinjection, sperms and urine-derived mesenchymal stem cell mitochondria are jointly injected into mature oocytes for blastaea. It is found that the urine-derived mesenchymal stem cell mitochondria extracted by the present disclosure can improve the quality of oocytes, increase the fertilization rate and embryo quality, and increase the development rate of human in-vitro fertilized embryos.

Further, the urine-derived mesenchymal stem cell mitochondria are autologous urine-derived mesenchymal stem cell mitochondria. The autologous urine-derived mesenchymal stem cell mitochondria and oocytes come from the same person, that is to say, the patient's own urine-derived mesenchymal stem cells, rather than a third-party source.

Specifically, the transplantation method comprises: the sperms are grabbed and braked using a micromanipulation needle, the sperms are pressed against the tip of the injection needle, transferred into the urine-derived mesenchymal stem cell mitochondrion droplets (the mitochondrion preservation solution containing the urine-derived mesenchymal stem cell mitochondria prepared by the above method), sucked for many times to form a homogenate, the urine-derived mesenchymal stem cell mitochondrion droplets are sucked, and the urine-derived mesenchymal stem cell mitochondrion droplets together with the sperms are injected into the cytoplasm of mature oocytes. After the injection is completed, the cytoplasm is transferred into embryo culture solution for blastocyst culture.

Further, the transplantation method comprises: under a micromanipulation table, sperms are grabbed and braked using a micromanipulation needle with a diameter of 4.5 um, the sperms are pressed against the tip of the injection needle, transferred into mitochondrion droplets and sucked for many times to form a homogenate, the urine-derived mesenchymal stem cell mitochondrion droplets are sucked using the injection needle, and 40,000-60,000 mitochondria at the 10 um volume length of the front end of the injection needle together with sperms are injected into the cytoplasm of mature oocytes. After injection is completed, the cytoplasm is transferred into the embryo culture solution for blastocyst culture.

Preferably, 48-52 thousand urine-derived mesenchymal stem cell mitochondria ((the urine-derived mesenchymal stem cell mitochondria extracted by the above method)) are injected into the cytoplasm of mature oocytes.

Provided is use of the transplantation method of the above urine-derived mesenchymal stem cell mitochondria in a drug for improving the quality of oocytes.

Specifically, 40,000-60,000 urine-derived mesenchymal stem cell mitochondria are injected into the cytoplasm of mature oocytes to improve the quality of oocytes.

Preferably, 40,000-60,000 autologous urine-derived mesenchymal stem cell mitochondria are injected into the cytoplasm of mature oocytes to improve the quality of oocytes. The autologous urine-derived mesenchymal stem cell mitochondria and oocytes come from the same person, that is to say, the patient's own urine-derived mesenchymal stem cells without involving the introduction of a third-party genetic material and ethical issues.

More specifically, during the intracytoplasmic sperm microinjection, sperms together with 40,000-60,000 autologous urine-derived mesenchymal stem cell mitochondria are injected into mature oocytes.

Further, the sperms are grabbed and braked using a micromanipulation needle and the sperms are pressed against the tip of the injection needle, transferred into the urine-derived mesenchymal stem cell mitochondrion droplets (the mitochondrion preservation solution containing the urine-derived mesenchymal stem cell mitochondria prepared by the above method) and sucked for many times to form a homogenate, the urine-derived mesenchymal stem cell mitochondrion droplets are sucked, and 40,000-60,000 autologous urine-derived mesenchymal stem cell mitochondrion droplets together with sperms are injected into the cytoplasm of mature oocytes.

Provided is a drug for improving the quality of oocytes, the drug comprising the urine-derived mesenchymal stem cell mitochondria.

The R & D team of the inventor comprehensively evaluated many autologous mesenchymal stem cells (bone marrow, fat and urine) from various levels in the aspects of mitochondrion function and metabolic capacity, and made safety verification. It was found that the urine-derived mesenchymal stem cell mitochondria are more similar to oocytes compared with other types of mesenchymal stem cells in terms of maturity, function and metabolic mode. In addition, the urine-derived mesenchymal stem cell mitochondria have the advantage of non-invasive acquisition, and are also suitable to be used as the autologous cell mitochondrion source. Therefore, it has multiple research and application advantages. Further, the inventor extracted the urine-derived mesenchymal stem cell mitochondria from the patient to be jointly injected together with sperms during the ICSI. It is found that the fertilization rate and embryo quality of test group are significantly improved.

Compared with the prior art, the present disclosure has the beneficial effects:

(1) The test shows that by adopting the method for extracting the urine-derived mesenchymal stem cell mitochondria, the urine-derived mesenchymal stem cell mitochondria with strong activity can be extracted from the urine of patients.

The present disclosure provides a transplantation method of urine-derived mesenchymal stem cell mitochondria. Through transplantation of the urine-derived mesenchymal stem cell mitochondria, the quality of oocytes is improved, and the fertilization rate and embryo quality of human in-vitro fertilization are significantly improved.

(2) The present disclosure provides a transplantation method of urine-derived mesenchymal stem cell mitochondria. The transplantation method can be used for in-vitro fertilization of low-prognosis patients with infertility during the traditional ICSI in combination with transplantation of urine-derived mesenchymal stem cell mitochondria, with a good treatment effect;

(3) The extracted urine-derived mesenchymal stem cell mitochondria from the patient's autologous source together with sperms are injected during the ICSI, from which it is found that the test group has a significant improvement in the aspects of fertilization rate and embryo quality;

(4) The present disclosure solves the problems of heterogeneity and ethics of allogeneic mitochondrion transplantation existing in the prior art. The autologous mitochondrion source does not involve the introduction of third-party genetic material and ethical issues; moreover, the autologous mitochondrion source is safe, non-invasive, unrestricted, and highly available, the transplantation method adopted by the present disclosure can realize autologous non-invasive treatment of infertility;

(5) The transplantation method of the urine-derived mesenchymal stem cell mitochondria of the present disclosure is simple to operate, strong in feasibility and non-invasively obtained. The transplantation method can be used for autologous mitochondrion treatment of other types of diseases, such as Parkinson's syndrome, heart disease, degenerative nerve muscle diseases, metabolic diseases and other diseases related to abnormal mitochondrion metabolism.

### BRIEF DESCRIPTION OF THE DRAWINGS

To more clearly illustrate the embodiments of the present disclosure or technical solution in the prior art, drawings required to be used in the embodiments or in the prior art will be simply described below. Obviously, drawings used in the following description are only some embodiments of the present disclosure, and other drawings can also be made by persons of ordinary skill in the art without creative efforts according to these drawings.

FIG. 1 shows comparison of mitochondrion biological activity of mature oocytes from low-prognosis patients with those of normal people;

FIG. 2 is a light microscope image of P1-generation urinary-derived mesenchymal stem cells according to example 3 of the present disclosure;

FIG. 3 shows flow cytometric identification of urine-derived mesenchymal stem cells;

FIG. 4 is a diagram showing activity identification of urine-derived mesenchymal stem cell mitochondria extracted according to example 3 of the present disclosure;

FIG. 5 shows a process of joint injection of sperms and autologous urine-derived mesenchymal stem cell mitochondria during the ICSI according to the present disclosure;

FIG. 6 shows comparison of fertilization situations of control group and test group on the first day after in-vitro fertilization according to the present disclosure;

FIG. 7 shows comparison of embryo development situations of control group and test group on the third day after in-vitro fertilization according to the present disclosure;

FIG. 8 shows comparison of embryo development situations of control group and test group on the fifth day after in-vitro fertilization according to the present disclosure;

FIG. 9 shows comparison of mitochondrion copy numbers of urine-derived, bone-marrow and adipose-derived mesenchymal stem cells and ovarian granular cells;

FIG. 10 shows comparison of extracellular acid production capacities (ECAR) of urine-derived, bone-marrow and adipose-derived mesenchymal stem cells and ovarian granular cells;

FIG. 11 shows comparison of oxygen consumptions (OCR) of urine-derived, bone-marrow and adipose-derived mesenchymal stem cells and ovarian granular cells; and

FIG. 12 is an expression map of electron transport chain genes encoded by the mitochondria of urine-derived, bone marrow, adipose-derived mesenchymal stem cells and ovarian granular cells.

### DETAILED DESCRIPTION OF THE EMBODIMENTS

To make the objective, technical solution and advantages of the present disclosure more clear, the technical solution of the present disclosure will be further described in detail through embodiments in combination with drawings. Obviously, the described embodiments are only a part of embodiments of the present disclosure but not all the embodiments. Based on the embodiments of the present disclosure, all other embodiments obtained by persons of ordinary skill in the art without creative efforts are included within the protective scope of the present disclosure.

The urine-derived mesenchymal stem cell isolation culture medium, the urine-derived mesenchymal stem cell amplification culture medium, the lysate, the mitochondrion extraction solution, pancreatin and a mitochondrion preservation solution used in the following examples are commercially available, in which the urine-derived mesenchymal stem cell isolation culture medium (Cat. No. AV-1501, Asia Vector); the urine-derived mesenchymal stem cell amplification culture medium (Cat. No. AV-1501-B, Asia Vector); the lysate (Cat. No. MITOISO2 component, Sigma); the mitochondrion extraction solution (product number MITOISO2 component, Sigma); the pancreatin (Sigma); the mitochondrion preservation solution (product number MITOISO2 component, Sigma).

### Example 1

In this example, the urine-derived mesenchymal stem cell mitochondria were extracted by using the following method:

Urine was collected into a vessel and centrifuged, supernatant was discarded, PBS buffer was added into the vessel for resuspension, the resulting solution was centrifuged again, the supernatant was discarded, cell precipitates were resuspended using the urine-derived mesenchymal stem cell isolation culture medium, inoculated to a gelatin-coated 6-well plate, and the 6-well plate was put into an incubator for primary culture; the culture medium was completely changed once after cell clones were formed; after the clones are fused into a blockbuster, pancreatin was used for digestion, the pancreatin was sucked after digestion, the USC amplification culture medium was used for resuspension, the resuspended solution was inoculated into a new 6-well plate, and marked as a P1 generation (subsequent passage and culture were the same as those in this step).

(2) the P1 generation obtained in step (1) was placed in the incubator to continue culture, digestion was conducted using pancreatin when the P1 generation grown to cover the 95% of area of the 6-well plate, the pancreatin was sucked after digestion, resuspension was conducted using the urine-derived mesenchymal stem cell amplification culture medium, resuspended solution was centrifuged, the supernatant was discharded, a cell lysis solution was added for lysis on ice, then a mitochondrion extraction solution was then added, and uniformly mixed; and the mixed solution was centrifuged, the supernatant was sucked, transferred into another vessel and centrifuged again, and the supernatant was discarded to obtain precipitates namely mitochondria; and

(3) the obtained mitochondria were centrifuged again and washed, the supernatant was discarded, and the mitochondrion precipitate was resuspended using a mitochondrion preservation solution, and stored at the temperature of less than of 4° C.

### Example 2

This example provides a transplantation method of urine-derived mesenchymal stem cell mitochondria, comprising: during the intracytoplasmic sperm microinjection, sperms and urine-derived mesenchymal stem cell mitochondria (mitochondrion solution droplets obtained by resuspending the mitochondrion precipitate using the mitochondrion preservation solution) were jointly injected into mature oocytes for blastula culture to improve the quality of oocytes, improve the fertility rate and embryo quality, and increase the embryonic development rate of human in-vitro fertilization.

### Example 3

The Mitochondrion Biological Activity of Mature Oocytes of Low-Prognosis Patients in this Example was Compared with that of Normal People

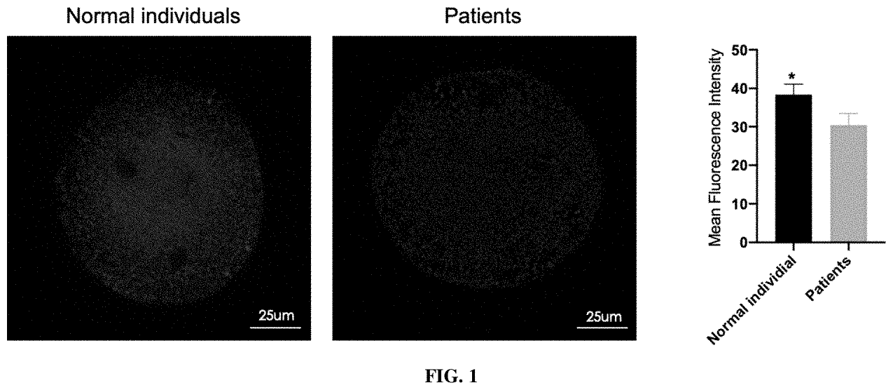

The mitochondrion activities of mature oocytes in normal people and low-prognosis patients were observed under a confocal microscope. TMRE is tetramethylrhodamine ethyl ester, namely, a mitochondrion membrane potential indicator. Red fluorescence represents the intensity of mitochondrion activity. The results are shown in FIG. 1.

It can be seen from the results from FIG. 1 that the biological activity of oocyte mitochondria in normal people is significantly higher than that in low-prognosis patients.

The Urine-Derived Mesenchymal Stem Cell Mitochondria from the Same Low Prognosis Patient were Extracted Through the Following Steps (1)-(3):

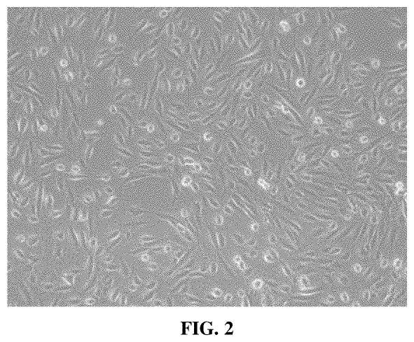

200 mL of urine from the above-mentioned low-prognosis patients was collected and dispensed into 50 mL sterile centrifuge tubes to be centrifuged at 1200 rpm for 10 min, supernatant was discarded, 20 mL of PBS was added for resuspension, the resulting resuspended solution was centrifuged again for 10 min at 1200 rpm, the supernatant was discarded, cell precipitates were resuspended using a USC isolation culture medium and then inoculated into a 0.1% gelatin-coated 6-well plate, the 6-well plate was placed in an incubator with 37° C. and 5% CO₂ for primary culture. The culture medium was completely changed once after small clones were formed on the 7th day, and 0.05% pancreatin was added for digestion after the clones were fused into a blockblaster (filled with the entire 100×microscope field of view) on the 14th day, the redundant pancreatin was sucked after digestion for 1 min, the rest solution was resuspended using the USC amplification culture medium and then inoculated into a new 6-well plate, and then the inoculated product was marked as the P1 generation (the light microscope graph of P1-generation urinary-derived mesenchymal stem cells is shown in FIG. 2).

Flow Cytometry Surface Marker Identification of Urine-Derived Mesenchymal Stem Cells

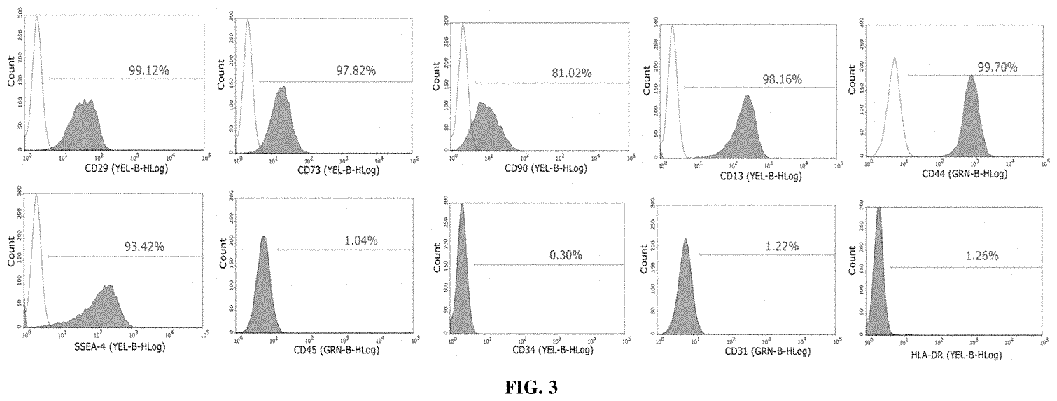

Urine-derived mesenchymal stem cells detected by flow cytometry positively expressed mesenchymal stem cell surface positive markers CD29, CD73, CD90, CD13, CD44 and SSEA-4, negatively expressed hematopoietic stem cells and endothelial cells such as CD45, CD34, CD31 and HLA-DR, proving that their mesenchymal origins. The result is shown in FIG. 3. It can be seen from the figure that the isolated cells of the present disclosure are urine-derived mesenchymal stem cells.

(2) the P1 generation urine-derived mesenchymal stem cells obtained in step (1) were put in the incubator with 37° C. and 5% CO₂ to continue culture, and the subsquent passage and culture are the same as them; pancreatin was used for digestion when the P1 generation grown to cover the 90% of area of the 6-well plate, the redundant pancreatin was sucked after digestion for 1 min, 1 mL of urine-derived mesenchymal stem cell amplification culture medium was used for resuspension, the supernatant was sucked to a 1.5 mL of sterile EP tube and centrifuged for 3 min at 1200 rpm, the supernatant was discarded, 500 ul of cell lysis solution was added for lysis on ice for 5 min (reversely and uniformly mixing every 1 min once), then 1 mL of mitochondrion extraction solution was added, and uniformly mixed; and the above mixed solution was centrifuged for 10 min at 5000 g, the supernatant was discarded to obtain precipitates namely mitochondria;

(3) the above mitochondria were centrifuged again at 5000 g for 10 min and washed, the supernatant was discarded, the precipitates were resuspended using 50 ul of mitochondrion preservation solution, and the resulting mitochondrion solution was stored on ice until use.

Urine-Derived Mesenchymal Stem Cell Mitochondria was Subjected to Activity Identification

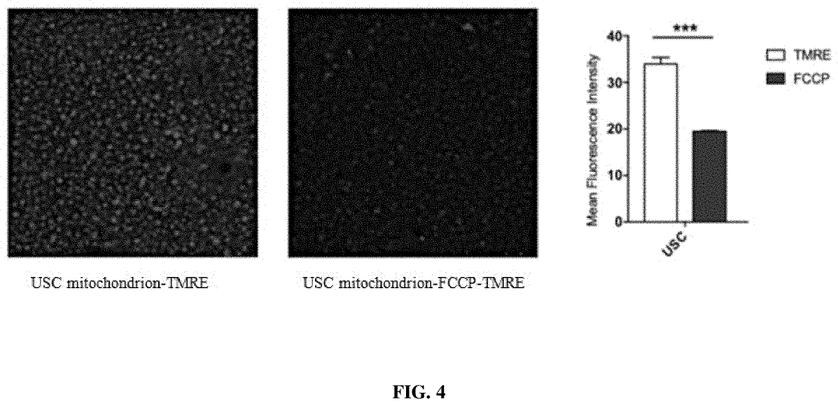

After the urine-derived mesenchymal stem cell mitochondria were extracted, the mitochondrion activity was observed under the confocal microscope. The results are shown in FIG. 4. In the figure, TMRE is tetramethylrhodamine ethyl ester, that is to say, TMRE is used as the mitochondrion membrane potential indicator, red fluorescence represents the intensity of mitochondrion activity; FCCP is an oxidative phosphorylation uncoupling agent. The left picture shows that TMRE, rather than FCCP, is added in the urine-derived mesenchymal stem cell mitochondria obtained in example 3, the red fluorescence intensity shows that the urine-derived mesenchymal stem cell mitochondria have activity; the right picture shows that TMRE and FCCP are added as a negative control, after that, the red fluorescence is significantly weakened and the mitochondrion activity is significantly reduced.

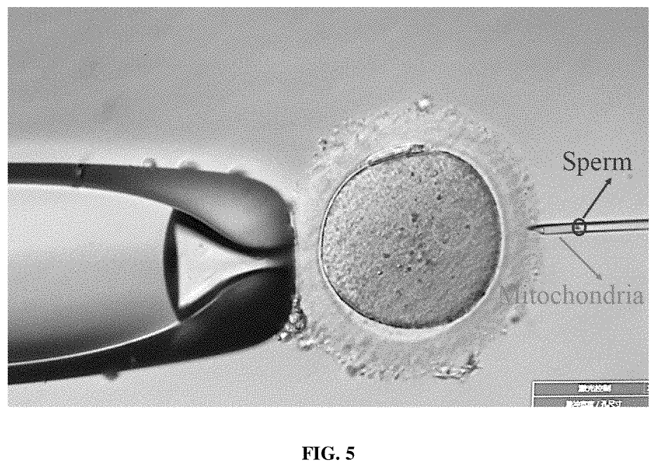

Then, mature oocytes were used to perform ICSI in-vitro fertilization, specifically: under the micromanipulation table, sperms were grabbed and braked using a micromanipulation needle with a diameter of 4.5 um, the sperms were pressed against the tip of the injection needle, transferred into mitochondrion droplets and repeatedly sucked to suck the mitochondrion droplets (from mitochondrion droplets obtained by resuspending mitochondrion precipitates using 50 ul of mitochondrion preservation solution in step (3)) to ensure the mitochondrion concentration required for injection, m volume length of the front end together with the previously grabbed and braked sperms were injected into the cytoplasm of mature oocytes (shown in FIG. 5), the mitochondria were located on the tip of the injector, the sperms were behind the mitochondria, and then the cytoplasm was transferred into the embryo culture medium for blastocyst culture.

The traditional ICSI group (control group) from the same patient's sister eggs was compared with test group of autologous USC mitochondrion transplantation during ICSI.

Test group: ICSI mitochondrion co-injection in-vitro fertilization was performed on the sister eggs from 3 patients using the method in example 3;

Control group: the control group is different from the test group in that the control group used the traditional ICSI in-vitro fertilization without autologous urine-derived mesenchymal stem cell mitochondrion transplantation; the specific method was as follows: mature oocytes were used for the traditional ICSI in-vitro fertilization, under the micromanipulation table, sperms were grabbed and braked using a micromanipulation needle with a diameter of 4.5 um, the sperms were pressed against the tip of the injection needle and then injected into the cytoplasm of mature oocytes, and then the cytoplasm was transferred into the embryo culture medium for blastocyst culture after injection was completed.

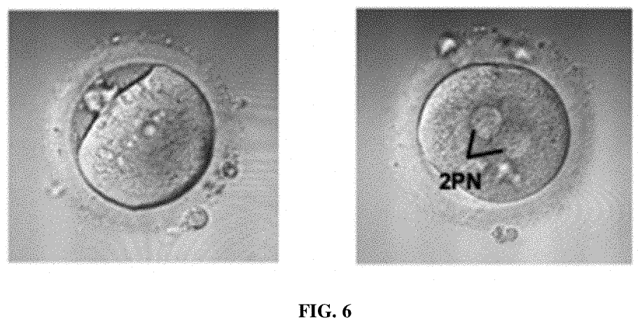

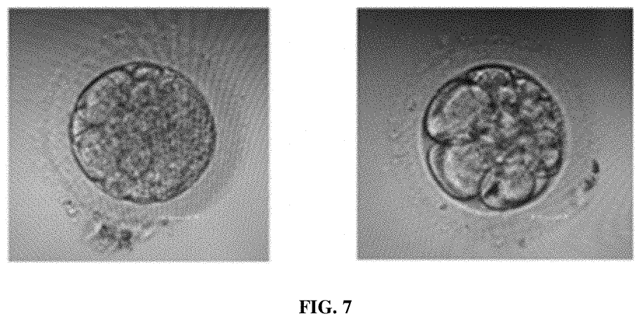

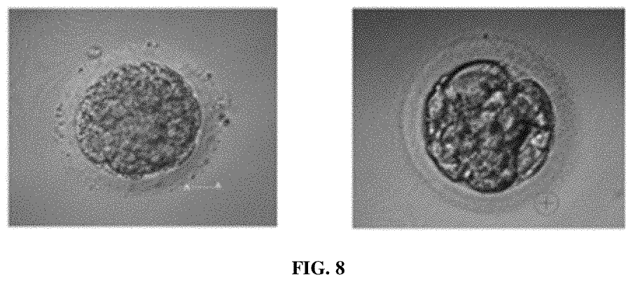

The fertilization situations and embryo situations of control group and test group were compared, and the results are shown in FIGS. 6-8. It can be seen from the figures that on the first day, the control group was fertilized abnormally; the test group was fertilized normally with double pronucleus (2PN) formed (FIG. 6); on the third day, the control group had grade IV embryos (fragments) and the test group had 9 cells Grade III embryos have normal cleavage speed (FIG. 7); on the fifth day, embryo development in the control group was blocked, and the test group was BC-grade early blastocysts (FIG. 8). The above test shows that the normal fertilization rate, cleavage speed, and embryo quality of the test group are all improved.

The inventors comprehensively evaluated many autologous mesenchymal stem cells (bone marrow, fat and urine) from various levels in the aspects of mitochondrion function and metabolic capacity, and made safety verification. It was found that the urine-derived mesenchymal stem cell mitochondria are more similar to oocytes compared with other types of mesenchymal stem cells in terms of maturity, function and metabolic mode, specifically:

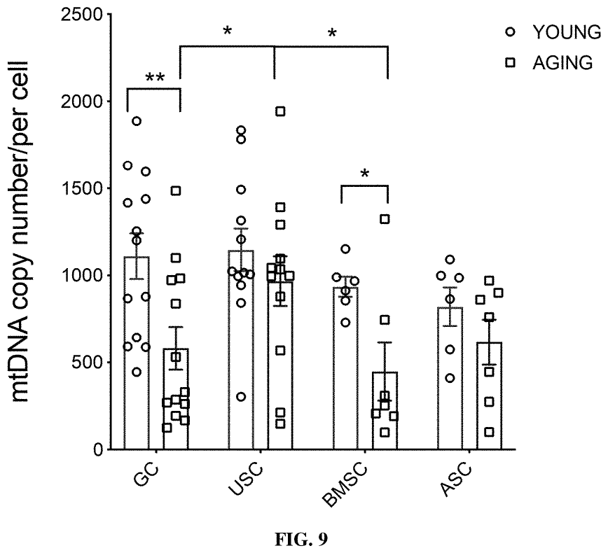

1. The mitochondrion copy numbers of urine-derived (USC), bone-marrow (BMSC), adipose (ASC)-derived mesenchymal stem cells and ovarian granular cells (GC) were compared, and the results are shown in FIG. 9.

It can be seen from FIG. 9 that there are no significant difference in the mitochondrion copy numbers of GC, USC, BMSC and ASC among young people; the mitochondrion copy numbers of GC and BMSC of the elderly are significantly reduced compared with those of young people, while the mitochondrion copy numbers of USC and ASC are not significantly reduced; the mitochondrion copy number of the elderly USC is significantly higher than that of the elderly GC and BMSC.

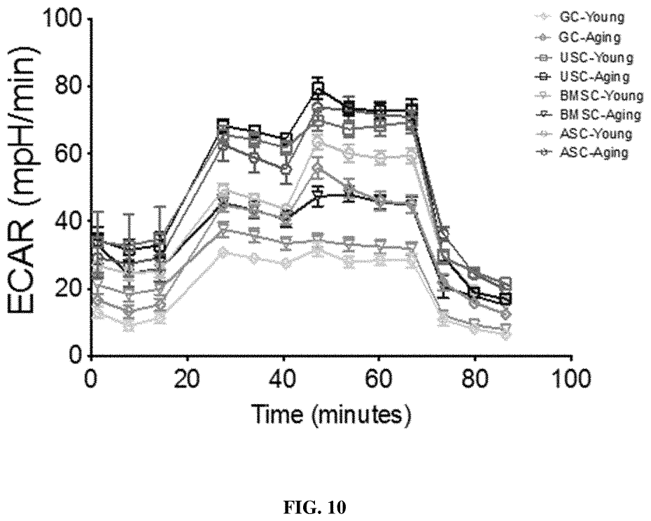

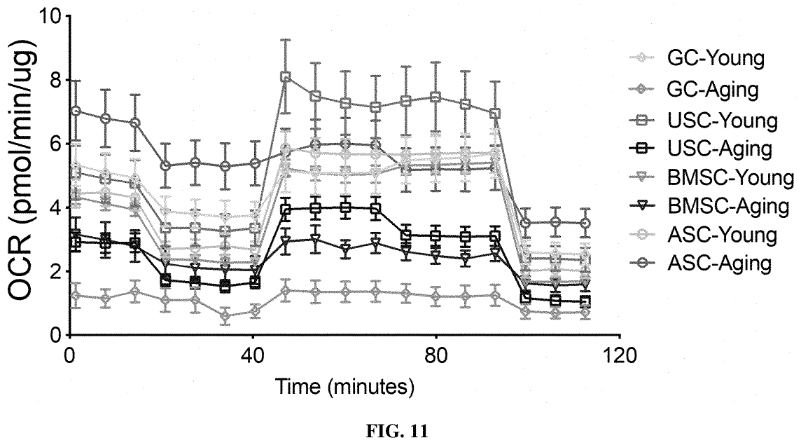

2. cell metabolism and mitochondrion functions of urine-derived (USC), bone marrow (BMSC), adipose (ASC)-derived mesenchymal stem cells and ovarian granular cells (GC) were compared, and the results are shown in FIG. 10 and FIG. 11. FIG. 10 shows extracellular acid production capability (ECAR) of the detected cells, and indirectly shows the cytosolic glycolysis capability; FIG. 11 shows the oxygen consumption (OCR) of the detected cells, which reflects the oxidative phosphorylation capacity of mitochondria.

It can be seen from FIG. 10 and FIG. 11 that the overall cell metabolism capability (including glycolysis and oxidative phosphorylation) of the urine-derived mesenchymal stem cells (USC) is higher than the overall cell metabolism capabilities of bone marrow, adipose-derived mesenchymal stem cells and ovarian granular cells.

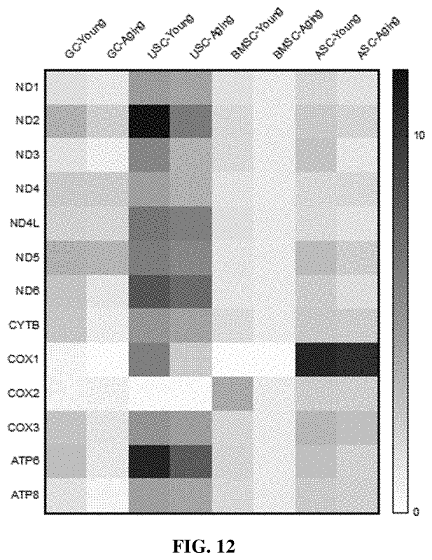

3. The expression patterns of the electron transport chain genes encoded by the mitochondria of urine-derived (USC), bone-marrow (BMSC), adipose (ASC)-derived mesenchymal stem cells and ovarian granulosa cells (GC) are shown in FIG. 12.

It can be seen from FIG. 12 that the expression level of the electron transport chain genes encoded by the urine-derived mesenchymal stem cell mitochondria (USC) is higher than that of other cell types in both young and old patients.

Through the above test, it can be seen that the urine-derived mesenchymal stem cell mitochondria have more application advantages than other types of mesenchymal stem cell mitochondria in terms of number, mitochondrion function, and gene expression pattern. In addition, the urine-derived mesenchymal stem cell mitochondria are suitably used as the first choice of the autologous mitochondrion source due to its characteristic of non-invasive and large-scale acquisition.

The above descriptions are only specific embodiments of the present disclosure, but the protective scope of the present disclosure is not limited thereto. Those skilled in the art can easily conceive that changes or substitutions should be included within the protective scope of the present disclosure. Therefore, the protective scope of the present disclosure shall be based on the protective scope of the claims.

What is claimed is:

1. Urine-derived mesenchymal stem cell mitochondrion, wherein the urine-derived mesenchymal stem cell mitochondria is extracted by the following method:
(1) collecting urine into a vessel, centrifuging, discarding supernatant, adding PBS buffer into the vessel for resuspension, centrifuging again, discarding the supernatant, resuspending cell precipitates using a urine-derived mesenchymal stem cell isolation culture medium, inoculating the resuspended cell precipitates into a gelatin-coated 6-well plate, and putting the 6-well plate into an incubator for primary culture; changing the culture medium once when cells clones are formed, digesting with pancreatin after the clones are fused into a blockbuster, sucking pancreatin after digestion, resuspending using a urine-derived mesenchymal stem cell amplification culture medium, and inoculating into a new 6-well plate, and marking a P1 generation;
(2) putting the P1 generation obtained in step (1) in the incubator to continue culture, digesting using pancreatin when the P1 generation grows to cover the 85%-95% of area of the 6-well plate, sucking the pancreatin after digestion, resuspending using the urine-derived mesenchymal stem cell amplification culture medium, centrifuging, discarding the supernatant, adding a cell lysis solution, lysing on ice, then adding a mitochondrion extraction solution, and uniformly mixing; and centrifuging, sucking the supernatant, transferring into another vessel, centrifuging again, and discarding the supernatant to obtain precipitates namely mitochondria; and
(3) centrifuging again, washing, discarding the supernatant, resuspending the mitochondrion precipitates using a mitochondrion preservation solution, and storing at 0-4° C.

2. The urine-derived mesenchymal stem cell mitochondria according to claim 1, wherein in step (1), the centrifuging is performed for 10 min at 120 rpm; the time for cell clone formation is 7 days, and the time of fusing the clones into blockbuster is 14 days.

3. The urine-derived mesenchymal stem cell mitochondria according to claim 1, wherein in step (1), the gelatin is a 0.1% gelatin aqueous solution; in step (1) and step (2), the incubators are all incubators with 37° C. and 5% CO₂; the pancreatin is a 0.05% pancreatin aqueous solution; and the digestion time is 1 min.

4. The urine-derived mesenchymal stem cell mitochondria according to claim 1, wherein in step (2), the lysis time is 5 min; the redundant pancreatin is sucked after digestion using pancreatin, 1 mL of urine-derived mesenchymal stem cell amplification culture medium is added into each well for resuspension, the above resuspension is transferred into a centrifuge tube to be centrifuged for 3 min at 1200 rpm, the supernatant is discarded, 500 ul of cell lysis solution is added to lyze the cells on ice for 5 min, and 1 ml of mitochondrion extraction solution is then added and uniformly mixed.

5. The urine-derived mesenchymal stem cell mitochondria according to claim 1, wherein in step (2), 1 ml of mitochondrion extraction solution is added and uniformly mixed; the above mixture is centrifuged for 10 min at 800 g, the supernatant is sucked and transferred to another centrifuge tube, and then centrifuged for 10 min at 5000 g, and the supernatant is discarded to obtain precipitates namely mitochondria.

6. A transplantation method of the urine-derived mesenchymal stem cell mitochondria according to claim 1, the transplantation method comprising: during the intracytoplasmic sperm microinjection, sperms and urine-derived mesenchymal stem cell mitochondria are jointly injected into mature oocytes; preferably, the urine-derived mesenchymal stem cell mitochondria are autologous urine-derived mesenchyme cytoplasmic stem cell mitochondria.

7. The transplantation method of the urine-derived mesenchymal stem cell mitochondria according to claim 6, the transplantation method comprising: the sperms are grabbed and braked using a micromanipulation needle, the sperms are pressed against the tip of the injection needle and transferred into a urine-derived mesenchymal stem cell mitochondrion droplets and sucked for many times to form a homogenate, then, the urine-derived mesenchymal stem cell mitochondrion droplets together with the previously grabbed and braked sperms are injected into the cytoplasm of mature oocytes.

8. The transplantation method of the urine-derived mesenchymal stem cell mitochondrion according to claim 7, the transplantation method comprising: under a micromanipulation table, sperms are grabbed and braked using a micromanipulation needle with a diameter of 4.5 um, the sperms are pressed against the tip of the injection needle, transferred into mitochondrion droplets and sucked for many times to form a homogenate, the urine-derived mesenchymal stem cell mitochondrion droplets are sucked using the injection needle, and 40,000-60,000 mitochondria at the 10 um volume length of the front end of the injection needle together with sperms are injected into the cytoplasm of mature oocytes.

9. A drug for improving the quality of oocytes, the drug comprising the urine-derived mesenchymal stem cell mitochondria according to claim 1.
